(19) United States
(12) Patent Application Publication
(10) Pub. No.: US 2022/0218671 A1
(43) Pub. Date: Jul. 14, 2022

(54) TELMISARTAN FOR THE TREATMENT OF CHRONIC KIDNEY DISEASE IN DOGS

(71) Applicants: BOEHRINGER INGELHEIM VETMEDICA GMBH, Ingelheim am Rhein (DE); University of Georgia Research Foundation, Inc., Athens, GA (US)

(72) Inventors: Anne Michelle TRAAS, Saint Joseph, MO (US); Amanda ERICKSON COLEMAN, Bogart, GA (US); Bianca Natália FERREIRA DE MOURA LOURENCO, Athens, GA (US); Kate Elizabeth CREEVY, College Station, TX (US); Scott Alan Brown, Winterville, GA (US)

(21) Appl. No.: 17/596,351

(22) PCT Filed: Apr. 30, 2020
(86) PCT No.: PCT/US2020/030579

§ 371 Date: Dec. 8, 2021

Related U.S. Application Data
(60) Provisional application No. 62/871,752, filed on Jul. 9, 2019.

(51) Int. Cl. A61K 31/4184; A61K 45/06; A61P 13/12; A61P 9/12

(52) U.S. Cl. CPC A61K 31/4184; A61P 9/12; A61P 13/12; A61K 45/06

(57) ABSTRACT

The present invention relates to telmisartan or a pharmaceutically acceptable salt thereof as a medicament for the treatment of elevated urinary protein-to-creatinine ratio (UPC) levels in dogs, wherein the therapeutically effective amount of telmisartan is administered in a daily dosage amount that is varied over a treatment period.

### FIELD OF THE INVENTION

The present invention relates to telmisartan or a pharmaceutically acceptable salt thereof as a medicament for the treatment of elevated urinary protein-to-creatinine ratio (UPC) levels in dogs, wherein the therapeutically effective amount of telmisartan is administered in a daily dosage amount that is varied over a treatment period.

### CROSS REFERENCE TO RELATED APPLICATIONS

This application is the national stage of International Application No. PCT/US2020/030579, filed Apr. 30, 2020, which claims the benefit of U.S. Provisional Application No. 62/871,752 filed Jul. 9, 2019, both of which are incorporated herein by reference.

### BACKGROUND OF THE INVENTION

It is estimated that 300,000 to 1 million of the 70 million pet dogs in the United States suffer from chronic kidney disease (CKD), a condition that also affects more than 10% of dogs of all breeds over 15 years of age presented to a university hospital [1-3]. Of these, approximately 52% are affected by glomerular lesions, the hallmark of which is abnormal protein loss in the urine (proteinuria) [4] in animals affected by CKD, proteinuria is considered a risk factor for the adverse outcomes of renal morbidity and mortality, as well as all-cause mortality; in fact, dogs with UPC>1.0 are approximately 3 times more likely to experience uremic crisis and death than those with UPC≤1.0 [5]. Importantly, these risks increase with the degree of proteinuria, and may even apply to patients with otherwise normal kidney function [5, 6]. Several canine studies, evaluating both clinical CKD patients [5] and utilizing experimental models [7] have shown an association between the presence of proteinuria and progression of CKD, as proteinuria is able to promote renal injury through several mechanisms [8].

For these reasons, intervention (in the form of dietary modification and treatment with pharmacologic agents designed to mitigate urinary protein loss) is considered standard-of-care for dogs with proteinuric CKD [9-11]. Interventions that reduce the magnitude of proteinuria in affected dogs are associated with improved outcomes [10, 12, 13]. Angiotensin-converting enzyme inhibitors (ACEI), such as enalapril, have been shown to decrease proteinuria in experimental [7] and naturally occurring [12, 14] models of canine CKD and are the drugs most widely prescribed for this purpose. By decreasing angiotensin II (ANG II) production, ACEI's hemodynamic effects are primarily via reduction of efferent glomerular arteriolar resistance, which lowers glomerular trans-capillary hydraulic pressure, thereby reducing the magnitude of proteinuria [15].

Despite their overall benefit in lowering proteinuria within populations, ACEI are not universally successful, with degree of anti-proteinuric effect varying considerably on a patient-to-patient basis. For example, in a clinical trial designed to evaluate the efficacy of enalapril as a treatment for naturally occurring proteinuria, a clinically significant (i.e., 50%) reduction in proteinuria was noted in only 9/14 (64%) subjects, with 3/14 (22%) experiencing an increase in proteinuria despite therapy with enalapril [14].

In a case study it has been reported that a 6 year-old female beagle had been administered with an initial dose of 0.43 mg/kg of body weight per day of telmisartan for 7 days, which had been increased to 0.86 mg/kg to treat refractory proteinuria [16].

In another case study, the successful management of refractory proteinuria and systemic hypertension in an 11-year old Yorkshire terrier with renal cell carcinoma with surgery, 0.43 mg/kg of telmisartan and 0.3 mg/kg of amlodipine has been described [17].

The International patent application WO 2019/008077 teaches an administration scheme of sartans for prophylaxis or treatment of hypertension in a cat, where the initial dosage is 1.0 to 5.0 mg/kg of bodyweight and is decreased in a subsequent period.

Protein Losing Nephropathies (PLN), commonly encountered in dogs with the finding of protein in the urine and quantified using a urine protein-creatinine ratio (UPC), include diseases, such as glomerulonephritis (GN), glomerulopathy, and amyloidosis. The normal glomerulus acts to allow filtration of small molecules but restricts passage of larger or negatively charged molecules. If the glomerulus is damaged, large or negatively charged molecules can pass through resulting in leakage into the urine. PLN may lead to CKD if the losses are not controlled. Controlling proteinuria is the primary focus for the treatment of PLN.

There is, therefore, a critical need for additional anti-proteinuric and sustainable options for canine patients suffering from proteinuric CKD and/or PLN.

### SUMMARY OF THE INVENTION

Now, it has been found that dogs can be treated against elevated urinary protein-to-creatinine ratio (UPC) levels by administering therapeutically effective amounts of telmisartan, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

Thus, one objective of the present invention consists in providing a new therapeutic approach for the treatment of dogs against elevated UPC levels.

Therefore, the invention relates to telmisartan or a pharmaceutically acceptable salt thereof for use in a method for the treatment of elevated UPC levels in a dog in need of such treatment, wherein the method comprises administration of a therapeutically effective amount of telmisartan to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

Furthermore, the invention relates to telmisartan or a pharmaceutically acceptable salt thereof as a medicament for the treatment of elevated urinary protein-to-creatinine ratio (UPC) levels, which are non-refractory to the treatment with ACE inhibitors in dogs.

In a further embodiment of the invention there is provided a method for the treatment of elevated urinary protein-to-creatinine ratio (UPC) levels in a dog in need of such treatment, wherein the method comprises administration of a therapeutically effective amount of telmisartan or a pharmaceutically acceptable salt thereof to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

In a further embodiment, the invention provides a method for the treatment of elevated urinary protein-to-creatinine ratio (UPC) levels, which are non-refractory to the treatment with ACE inhibitors in dogs, which method comprises administration of a therapeutically effective amount of telmisartan or a pharmaceutically acceptable salt thereof to a dog in need of such a treatment.

Furthermore the invention relates to a pharmaceutical composition for use in a method for the treatment of chronic kidney disease of elevated urinary protein-to-creatinine ratio (UPC) levels, in a dog in need of such treatment, which comprises telmisartan or a pharmaceutically acceptable salt thereof according to the invention and a pharmaceutically acceptable carrier.

### BRIEF DESCRIPTION OF THE DRAWINGS

FIG. 1 is a plot of the proportion of telmisartan-treated dogs compared to enalapril-treated dogs, which experienced a reduction of UPC 50% at day 30 (cp. Example 1).

FIG. 2 is a plot of the average change from baseline in UPC from day 30 to day 90 for telmisartan treated dogs compared to enalapril treated dogs (cp. Example 2).

FIG. 3 depicts the average percent change from baseline of UPC in telmisartan treated dogs compared to enalapril treated dogs from day 30 to day 120 (cp. Example 2), where at day 90 enalapril has been added to the telmisartan group and telmisartan has been added to the enalapril group.

FIG. 4 depicts the average change from baseline of UPC in telmisartan dogs compared to enalapril treated dogs from day 30 to day 120 (cp. Example 2), where at day 90 enalapril has been added to the telmisartan group and telmisartan has been added to the enalapril group.

FIG. 5 depicts the average change from baseline of UPC in telmisartan dogs compared to enalapril treated dogs from day 30 to day 120 (cp. Example 2), where at day 90 no enalapril has been added to the telmisartan group and vice versa.

### DETAILED DESCRIPTION OF THE INVENTION

Before the embodiments of the present invention it shall be noted that as used herein and in the appended claims, the singular forms “a”, “an”, and “the” include plural reference unless the context clearly dictates otherwise. Thus, for example, reference to “a preparation” includes a plurality of such preparations, reference to the “carrier” is a reference to one or more carriers and equivalents thereof known to those skilled in the art, and so forth. Unless defined otherwise, all technical and scientific terms used herein have the same meanings as commonly understood by one of ordinary skill in the art to which this invention belongs. All given ranges and values may vary by 1 to 5% unless indicated otherwise or known otherwise by the person skilled in the art, therefore, the term “about” was omitted from the description. Although any methods and materials similar or equivalent to those described herein can be used in the practice or testing of the present invention, the preferred methods, devices, and materials are now described. All publications mentioned herein are incorporated herein by reference for the purpose of describing and disclosing the substances, excipients, carriers, and methodologies as reported in the publications which might be used in connection with the invention.

Nothing herein is to be construed as an admission that the invention is not entitled to antedate such disclosure by virtue of prior invention.

The solution to the above technical problem is achieved by the description and the embodiments characterized in the claims.

In accordance with the present invention, methods are described herein for the treatment of elevated UPC levels (also referred to proteinuria) in a dog in need of such treatment, where the methods comprise administration of a therapeutically effective amount of telmisartan to the dog, the therapeutically effective amount of telmisartan being administered in a daily dosage amount that is varied over a treatment period starting with an initial dose of at least 1.0 mg/kg of bodyweight. For example, the daily dosage amount of telmisartan for a first period of time during the treatment period can be 1.0 to 1.5 mg/kg of body weight, where the daily dosage amount of telmisartan is increased for a second period of time subsequent the first period of time during the treatment period.

As used herein, the term “pharmaceutically acceptable salts” includes the metal salts or the addition salts which can be used in dosage forms. For example, the pharmaceutically acceptable salts of the compounds provided herein can be acid addition salts, base addition salts or metal salts, and can be synthesized from parent compounds containing a basic or acid residue by means of conventional chemical processes. Such salts are generally prepared, for example, by reacting the free acid or base forms of these compounds with a stoichiometric amount of the suitable base or acid in water or in an organic solvent or in a mixture of both. Non-aqueous media are generally preferred, such as ether, ethyl acetate, ethanol, isopropanol, or acetonitrile. Examples of acid addition salts include mineral acid additions salts such as, for example, hydrochloride, hydrobromide, hydroiodide, sulfate, nitrate, phosphate, organic acid addition salts such as, for example, acetate, maleate, fumarate, citrate, oxalate, succinate, tartrate, malate, mandelate, methanesulfonate and p-toluenesulfonate. Examples of alkali addition salts include inorganic salts such as, for example, ammonium salts and organic alkaline salts such as, for example, diethylamine, ethylenediamine, ethanolamine, N,N-dialkylenethanolamine, triethanolamine, glutamine and basic amino acid salts. Examples of metal salts include, for example, sodium, potassium, calcium, magnesium, aluminium and lithium salts.

As used herein, the term “pharmaceutically acceptable” relates to molecular entities and compositions that are physiologically tolerable and do not normally cause an allergic reaction or a similar adverse reaction, such as gastric discomfort, dizziness and the like, when administered to humans. As used herein, the term “pharmaceutically acceptable” preferably means that it is approved by a regulatory agency of the federal or state government or listed in the US pharmacopoeia or another pharmacopoeia, generally recognized for its use in animals, preferably in mammals and more particularly in dogs.

The term “proteinuria” as used herein embraces any kind of elevated, pathologic UPC levels, which can be pre-glomerular, glomerular, or post-glomerular in origin. Pathologic proteinuria is a persistent problem from glomerular damage, whereas functional proteinuria is generally transient. Infection or inflammation (including neoplasia) of the lower urinary tract can induce significant proteinuria, and urinary protein should always be evaluated in light of the urinary sediment and culture results and the clinical signs present. Non-glomerular renal diseases, such as pyelonephritis, severe chronic renal failure, or acute tubular necrosis may also cause proteinuria. Excessive protein delivery to the kidney (“pre-glomerular proteinuria”) may lead to proteinuria, in conditions such as hemoglobinuria or multiple myeloma. Proteinuria with elevated or pathologic UPC levels may be associated with chronic kidney disease (CKD) and/or protein losing nephropathy (PLN).

As used herein, the term “non-refractory to the treatment with ACE inhibitors” refers to dogs suffering from proteinuria, which can be treated with an ACE inhibitor, but with less efficacy than telmisartan. To the contrary the elevated level of UPC of dogs that are refractory to ACE inhibitors cannot be lowered with the aid of ACE inhibitors.

In the non-refractory sub-population of dogs the efficacy of treatment with an ACE inhibitor is 10%, 15%, 20%, 25%, 30%, 35%, 40%, 45%, 50%, more than 50%, more than 60%, or more than 70% less effective than telmisartan for lowering their UPC levels.

In a preferred embodiment the telmisartan and/or the method according to the invention relates to the treatment of the non-refractory subpopulation of dogs. However, the administration scheme according to the invention may advantageously be administered to both the subpopulations, the non-refractory as well as to refractory dogs.

The dogs to be treated with telmisartan according to the invention are preferably pet dogs of any breed including any kind of mongrel. Depending on the size of the breed or mongrel they will suffer from proteinuria with elevated UPC levels at an age of 7 years or more, preferably from 7 to 18 years, in particular from 8 to 16 years. Small breeds will as a rule suffer at a later age, preferably from 8 to 18, from this disease than big ones, which may be affected at an age of 7 to 16 years.

As used herein, the terms “together with” or “in combination with” covers both separate and sequential administration of telmisartan and another drug. For example, when the agents are administered sequentially, either the telmisartan or the other drug may be administered first. When administration is simultaneous, the agents may be administered either in the same or a different pharmaceutical composition. Adjunctive therapy, i.e. where one agent is used as a primary treatment and the other agent is used to assist that primary treatment, is also an embodiment of the present invention.

In one embodiment of the present invention, adjunctive therapy includes adding the other agent to telmisartan after a period of time such as after 30 days, after 40 days, after 60 days, after 70, 80, 90 days, 100 days, 110 days, 120 days, or after 1, 2, 3, 4, 5, 6, or 12 months, or after any period of time between 30 and 120 days, or after any period of time between 1 and 12 months. In one embodiment, the adjunctive therapy is started at day 90. In one embodiment, the other agent is enalapril. In one embodiment, the primary agent is telmisartan, the other agent is enalapril, and the other agent is added to the telmisartan treatment at 90 days, or after 90 days.

In yet another embodiment, the primary agent is telmisartan, the other agent is selected from the group consisting of amlodipine, pimobendan, levosimendan, ramipril, benazepril and enalapril, and the other agent is added to treatment with telmisartan at 90 days, 120 days, 180 days, 360 days, or at any time between 90 days and 360 days.

The one or more active ingredients may be used either as separate formulations or as a single combined formulation. When combined in the same formulation it will be appreciated that the two compounds must be stable and compatible with each other and the other components of the formulation.

Formulations of the invention include those suitable for oral, parenteral (including subcutaneous e.g. by injection or by depot tablet, intradermal, intrathecal, intramuscular e.g. by depot and intravenous), rectal and topical (including dermal, buccal and sublingual) or in a form suitable for administration by inhalation or insufflation administration. The most suitable route of administration may depend upon the condition and disorder of the patient. Preferably, the compositions of the invention are formulated for oral administration.

The formulations may conveniently be presented in unit dosage form and may be prepared by any of the methods well known in the art of pharmacy e.g. as described in “Remington: The Science and Practice of Pharmacy”, Lippincott Williams and Wilkins, 21^{st} Edition, (2005). Suitable methods include the step of bringing into association to active ingredients with a carrier which constitutes one or more excipients. In general, formulations are prepared by uniformly and intimately bringing into association the active ingredients with liquid carriers or finely divided solid carriers or both and then, if necessary, shaping the product into the desired formulation. It will be appreciated that when the two active ingredients are administered independently, each may be administered by a different means.

Formulations suitable for oral administration may be presented as discrete units such as capsules, cachets or tablets, in particular chewable tablets, each containing a predetermined amount of active ingredient; as powder or granules; as a solution or suspension in an aqueous liquid or non-aqueous liquid; or as an oil-in-water liquid emulsion or water-in-oil liquid emulsion. The active ingredients may also be presented a bolus, electuary or paste.

Alternatively, the active ingredients may be incorporated into oral liquid preparations such as aqueous or oily suspensions, solutions, emulsions, syrups or elixirs. Formulations containing the active ingredients may also be presented as a dry product for constitution with water or another suitable vehicle before use. Such liquid preparations may contain conventional additives such as suspending agents (e.g. sorbitol syrup, methyl cellulose, glucose/sugar syrup, gelatin, hydroxymethyl cellulose, carboxymethyl cellulose, aluminium stearate gel and/or hydrogenated edible fats), emulsifying agents (e.g. lecithin, sorbitan mono-oleate and/or acacia), non-aqueous vehicles (e.g. edible oils, such as almond oil, fractionated coconut oil, oily esters, propylene glycol and/or ethyl alcohol), and preservatives (e.g. methyl or propyl p-hydroxybenzoates and/or sorbic acid).

In addition, the oral formulation may contain one or more flavoring agents, which enhance the compliance of the dog to be treated to chew and swallow the medication.

Most preferably telmisartan is administered orally in form of a chewable tablet or as an aqueous solution containing benzalkonium chloride as in the product Semintra®, which is commercially available from Boehringer Ingelheim Vetmedica GmbH, Ingelheim Germany.

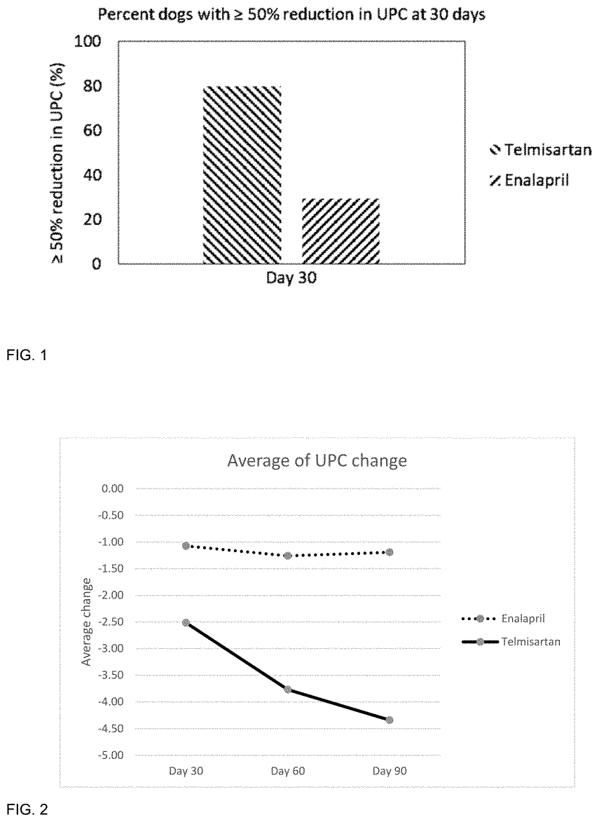

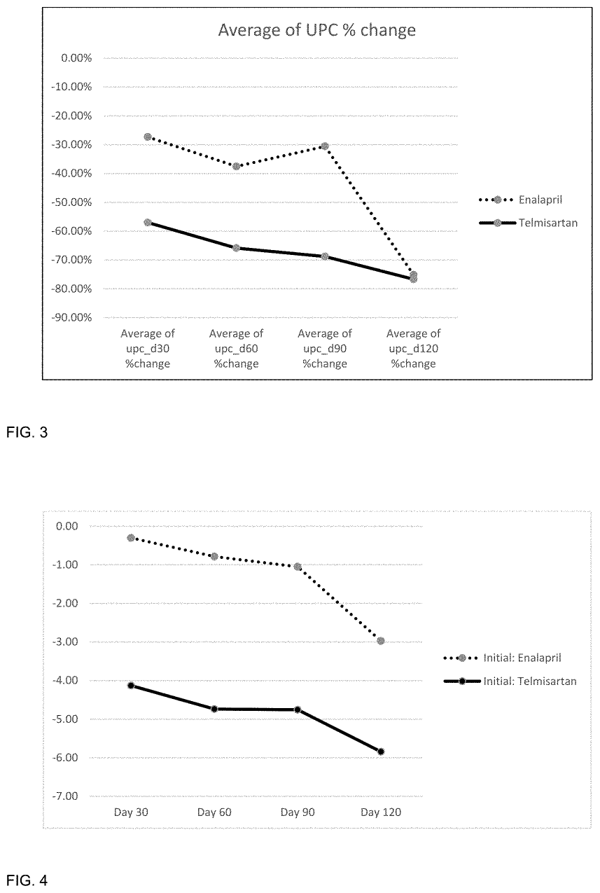

In particular, the following items are disclosed herein:
a) Telmisartan or a pharmaceutically acceptable salt thereof for use in a method for the treatment of elevated urinary protein-to-creatinine ratio (UPC) levels in a dog in need of such treatment, wherein the method comprises administration of a therapeutically effective amount of telmisartan to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.
b) Telmisartan according to item a), wherein the elevated UPC levels are associated with chronic kidney disease (CKD), protein losing nephropathy (PLN) and/or systemic hypertension.
c) Telmisartan according to item a) or b), which is the sodium or potassium salt thereof.
d) Telmisartan according to any one of items a) to c), wherein the daily therapeutically effective amount thereof ranges from 1.0 to 4.0 mg/kg, preferably 1.0 to 3.5 mg/kg, in particular 1.0 to 3.0 mg/kg of body weight.
e) Telmisartan according to any one of items a) to d), wherein the daily dosage amount of telmisartan is increased for the second period of time by an incremental amount ranging from 0.25 to 2.50 mg/kg of body weight.
f) Telmisartan according to any one of items a) to e), wherein the daily dosage amount of telmisartan for a first period of time during the treatment period is 1.0 to 1.5 mg/kg of body weight, and the daily dosage amount of telmisartan for the second period of time is 1.75 to 3.50 mg/kg of body weight.
g) Telmisartan according to any one of items a) to f), wherein the daily dosage amount of telmisartan is decreased after the second period of time by an incremental amount ranging from 0.25 to 2.50 mg/kg of body weight.
h) Telmisartan according to any one of items a) to g), wherein the daily dosage amount of telmisartan is decreased after the second period of time when the urinary protein-to-creatinine ratio (UPC) level measured for the dog decreases by at least 70% in relation to a baseline UPC value measured for the dog prior to the first period of time.
i) Telmisartan according to any one of items a) to h), which is administered together with at least one other drug to a dog in need of such a treatment.
j) Telmisartan according to item i), wherein the other drug is selected from the group consisting of calcium channel blockers, preferably amlodipine, cardiotonic-calcium sensitizing agents, preferably pimobendan or levosimendan, ACE inhibitors, preferably ramipril, benazepril or enalapril.
k) Telmisartan according to any one of items a) to j), wherein the UPC level is decreased by at least 50% compared to the baseline within the first period of treatment.
l) Telmisartan or a pharmaceutically acceptable salt thereof as a medicament for the treatment of diseases or disorders associated with elevated urinary protein-to-creatinine ratio (UPC) levels, which are non-refractory to the treatment with ACE inhibitors in dogs.
   A significantly greater proportion of telmisartan-treated dogs compared to enalapril-treated dogs, shows at least a 50% reduction in UPC at day 30 (16/20 (80%) versus 5/17 (29.4%), respectively) as shown in FIG. 1.
   As shown in FIG. 2 the average change in UPC is greater from day 30 to day 90 for telmisartan treated dogs compared to enalapril treated dogs with a greater change realized at day 90 for telmisartan treated dogs. In addition, the average percent change from baseline in UPC is greater in telmisartan treated dogs compared to enalapril treated dogs from day 30 to day 90 as shown in FIG. 3. The combination of telmisartan and enalapril from day 90 to day 120 achieves a reduction of more than 70 of the average UPC.
m) A method for the treatment of elevated urinary protein-to-creatinine ratio (UPC) levels, in a dog in need of such treatment, wherein the method comprises administration of a therapeutically effective amount of telmisartan or a pharmaceutically acceptable salt thereof to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.
n) The method according to item m), wherein the elevated UPC levels are associated with chronic kidney disease (CKD), protein losing nephropathy (PLN) and/or systemic hypertension.
o) The method according to item m), which comprises administration of an effective amount of the sodium or potassium salt of telmisartan.
p) The method according to item m), wherein the daily therapeutically effective amount of telmisartan ranges from 1.0 to 4.0 mg/kg, preferably 1.0 to 3.5 mg/kg, in particular 1.0 to 3.0 mg/kg of body weight.
q) The method according to item m), wherein the daily dosage amount of telmisartan is increased for the second period of time by an incremental amount ranging from 0.25 to 2.50 mg/kg of body weight.
r) The method according to item m), wherein the daily dosage amount of telmisartan is decreased after the second period of time by an incremental amount ranging from 0.25 to 2.50 mg/kg of body weight.
s) The method according to item r), wherein the daily dosage amount of telmisartan is decreased after the second period of time when the urinary protein-to-creatinine ratio (UPC) level measured for the dog decreases by at least 70% in relation to a baseline UPC value measured for the dog prior to the first period of time.
t) The method according to item m), wherein the method further comprises administration of at least one other drug to such dog in need of such a treatment.
u) The method according to item t), wherein the other drug is selected from the group consisting of calcium channel blockers, cardiotonic-calcium sensitizing agents and ACE inhibitors.
v) The method according to item u), wherein the other drug is selected from the group consisting of amlodipine, pimobendan, levosimendan, ramipril, benazepril and enalapril.
w) The method according to item m), wherein the UPC level is decreased in by at least 50 compared to the baseline within the first period of treatment.
x) A method for the treatment of diseases or disorders, which are associated with elevated urinary protein-to-creatinine ratio (UPC) levels, which are non-refractory to the treatment with ACE inhibitors in dogs, which method comprises administration of a therapeutically effective amount of telmisartan or a pharmaceutically acceptable salt thereof to a dog in need of such a treatment.

The invention now being generally described, will be more readily understood by reference to the following Examples, which are included merely for purposes of illustration of certain aspects and embodiments of the present invention, and are not intended to limit the invention.

### EXAMPLES

Experimental Methods and Design. A prospective, block-randomized, double-blind clinical trial has been carried out. Fifty-four client-owned dogs with persistent pathologic renal proteinuria have been recruited over a 2-year period.

### Example 1

Animals. Azotemic and non-azotemic dogs (N=54) with hypertensive and non-hypertensive CKD have been recruited prospectively from patients presented to the hospital. Dogs included as cases will have confirmed persistent pathologic renal proteinuria due to CKD; in order to be classified as such, fulfillment of the criteria described below will be required.

Inclusion criteria. Included animals had an UPC level of approximately 2.0 (for non-azotemic patients; IRIS stage 1) or approximately 0.5 (for azotemic patients; IRIS stages 2-4), documented in each of two urine samples collected 2 weeks apart. Abdominal ultrasound findings consistent with CKD (bilaterally small, irregular kidneys) and absence of renal neoplasia have also been documented.

Exclusion criteria. Animals have been excluded if one or more of the following are identified: evidence of hemorrhage, inflammation or bacteria on urine sediment analysis; positive urine culture at the time of identification of proteinuria; positive heartworm antigen test within 3 months of identification of proteinuria and/or not currently receiving regular monthly heartworm preventive; historical, physical examination or clinical pathologic findings suggestive of acute kidney injury, infectious nephropathy or lower urinary tract infection; systolic hypotension (SBP<120 mm Hg); moderate-to-severe hyperkalemia (serum K>6.5 mmol/L); history of having received oral ACEi and/or corticosteroids in the month (ACEi) or 2 weeks (corticosteroids) preceding examination; concurrent illness associated with proteinuria, the treatment of which might result in mitigation of proteinuria (e.g, systemic lupus erythematosis, ehrlichiosis, neoplasia). Dogs with suspected or confirmed hyper-adrenocorticism and diabetes mellitus have been included if their disease is considered well controlled with medical therapy.

Patient grouping for block randomization: Once included in the study and based on the presence/degree of azotemia, dogs have been grouped according to the International Renal Interest Society (IRIS) classification scheme for CKD. Those classified as IRIS stages 2-4 (serum creatinine ≥1.4 mg/dL with inappropriately dilute urine [USG<1.030]) have been considered azotemic (AZ), and those classified as IRIS stage 1 (creatinine <1.4 mg/dL) have been considered non-azotemic (non-AZ). Within each of these two groups, dogs will then be stratified according to IRIS recommendations for arterial pressure (AP) substaging. According to this scheme, dogs with persistent average indirect arterial systolic BP<150 mm Hg will be classified as AP0 (minimal risk for target organ damage). Those with persistent average indirect arterial systolic BP150 mm Hg have been classified as AP1-3 (at risk for target organ damage). Four groups will thus be identified:

1. AZ (IRIS Stages 2-4), IRIS substage AP1-3 3. non-AZ (IRIS Stage 1), IRIS substage AP1-3

2. AZ (IRIS Stages 2-4), IRIS substage AP0 4. non-AZ (IRIS Stage 1), IRIS substage AP0

Once placed into one of these four groups, each patient has then been assigned, based on a randomized blocking scheme, to receive either enalapril (n=27) or telmisartan (n=27), as described below, with the aim of grouping being to ensure that equal numbers of each are included into the two treatment groups.

Baseline. On inclusion (day 0), all owners have been required to read/sign a form consenting to their pets' participation in the study. The following baseline data have been collected for each case: full physical examination (performed by one of the study investigators), fundic examination, blood pressure measurement, serum chemistry panel, urinalysis, abdominal ultrasound, UPC and urine culture. The results of screening tests, if performed within 2 weeks of inclusion in the study, may be used as baseline information. Baseline UPC has been defined as the average of two measurements, taken 2 weeks apart, prior to enrollment.

ARB/ACEi therapy. On day 0, each dog has been randomized to receive telmisartan at 1 mg/kg PO q 24 h (TEL group, n=27) or enalapril at 0.5 mg/kg PO q 12 h (ENAL group, n=27) in a double-blind manner. Randomization and dispensation of telmisartan or enalapril has been carried out at the appropriate doses. Owners have been provided with appropriate contact numbers in the event of an emergency. Enalapril is readily available and telmisartan has been provided by Boehringer Ingelheim Vetmedica Inc., St. Joseph, Mo. in form of the an aqueous solution, which is commercially available as Semintra®.

Antihypertensive/other therapy. For dogs that are classified as AP3 (SBP180 mmHg; 200 mmHg in sighthounds), a calcium channel blocker (CCB; amlodipine, 0.1 mg/kg PO q 24 hours) has been administered contemporaneously. Co-administration of RAAS-inhibitors and CCB is common in human patients, recommended by a panel of veterinary experts²² and shown to be efficacious in a laboratory model of proteinuria. All dogs have been started or maintained on a commercially available diet formulated to be low in phosphorus and protein, for at least 1 month prior to enrollment. During the study period, diet remained constant. Treatment with fish oil has been allowed, provided that the dog has been receiving this supplement for >1 month at the time of enrollment.

Monitoring: The monitoring protocol followed the recommendations of the IRIS Canine GN Study Group Standard Therapy Subgroup. All dogs have been rechecked on day 7, at which time physical examination, SBP, serum creatinine (sCr) and serum potassium (K) have been evaluated. An increase in sCr of >30% compared to baseline or identification of moderate/severe hyperkalemia (serum K>6.5 mmol/L) or systolic hypotension (SBP<120) has prompt the investigator unmasking and removal of the patient from the study. For dogs in which average SBP of approximately 180 mm Hg was reliably identified (i.e. dogs classified as AP3), amlodipine will be up-titrated to 0.1 mg/kg PO BID. Thereafter, dogs classified as AP3 have been rechecked at 7-day intervals to ensure efficacy of therapy with adjustment of antihypertensive therapy. At each visit, if average SBP measurements remained at about 180 mm Hg, then the dog's amlodipine dose have been increased in increments of 0.05 mg/kg BID to a maximum dose of 0.3 mg/kg BID. SBP and sCr have been rechecked 7 d following any adjustments.

Final phase I visit. On day 30, all dogs have undergone physical examination, SBP, serum biochemistry, urinalysis and UPC measurement. At this and all subsequent time points, urine for UPC measurement will consist of a pooled sample, created by combining three free-catch specimens collected and refrigerated by the owner on the preceding day.

Major objective endpoints. The major objective endpoints of phase I are percentage change in UPC (ΔUPC) and percentage of patients achieving 50% reduction or decrease to <0.5 of UPC after 30 d of therapy.

Conclusions. A significantly greater proportion of telmisartan-treated dogs compared to enalapril-treated dogs, experienced 50% reduction in UPC at day 30 (16/20 (80%) versus 5/17 (29.4%), respectively; P=0.003) as shown in FIG. 1.

### Example 2

Specific Objectives #2 and #3 (Phases II and III; Intermediate-Term Phases)

Phase II of this study compared the efficacy of enalapril and telmisartan when these drugs were used as part of protocols that allow their up-titration, and phase III will evaluate their combination in dogs whose proteinuria persisted in the face of the highest doses of each drug alone. Each of the 54 dogs will remain in the treatment group to which he/she was assigned in phase I. Within these groups, up-titration of study drugs, followed by combination therapy have been performed if proteinuria persisted with UPC at about 0.5 on monthly rechecks.

ARB/ACEi Therapy.

Phase II (days 31-90): For those dogs in which UPC<0.5 was identified on day 30, treatment continued with telmisartan at a dose of 1 mg/kg PO q 24 h or enalapril at a dose of 0.5 mg/kg PO BID until the end of the study (day 120). For those in which UPC-0.5 was identified on day 30, the dose of study drug has been up-titrated monthly in increments of 1 mg/kg PO q 24 h (TEL group) or 0.5 mg/kg BID (ENAL group) until a target UPC<0.5 was attained OR a “ceiling dose” (3 mg/kg PO q 24 h for telmisartan or 1.5 mg/kg PO BID for enalapril) of either drug is reached, whichever occurs first.

Phase III (days 91-120): For those dogs in which UPC<0.5 was identified on or before day 90, treatment continued with telmisartan or enalapril at the dose producing proteinuria control until the end of the study. For those in which UPC approximately 0.5 was identified on day 90, enalapril at a dose of 0.5 mg/kg BID or telmisartan at a dose of 1 mg/kg q 24 h has been added for dogs in the TEL and ENAL groups, respectively. Combination therapy continued for 1 month, until the end of the study.

Monitoring. If a change was made to an individual dog's treatment regimen on day 30, he/she has been rechecked one week later (day 37), at which time SBP, sCr and serum K levels have been evaluated. An increase in creatinine of >30% or identification of moderate/severe hyperkalemia (serum K>6.5 mmol/L) prompted the investigator unmasking and removal of the patient from the study. If mild hyperkalemia (serum K of 6.1-6.5 mmol/L) was identified, up-titration to the next dose has not been performed, regardless of UPC.

Thereafter, persistently proteinuric dogs have been monitored monthly (i.e., on days 60, 90) by means of SBP, UPC and urinalysis. Urine culture have been performed if active urinary sediment was identified. For dogs in which proteinuria persisted and up-titration of drug was required, SBP, sCr and serum K have been rechecked one week after adjustments (days 67, 97), with criteria for unmasking and further dose up-titration as outlined above. Dogs in which UPC<0.5 was identified at any time point have undergone recheck of monitoring parameters at the conclusion of the study only (d 120).

Final visit: On day 120, all dogs have undergone full physical examination, SBP, serum renal biochemistry, urinalysis (cystocentesis) and UPC measurement.

Major objective endpoints. The major objective endpoints for phase II included AUPC from baseline and percentage of patients achieving 50% reduction or decrease to <0.5 of UPC following a total of 3 months of therapy, as well as time to 50% reduction or decrease to <0.5 of UPC. Phase III's major objective endpoints included AUPC from baseline, AUPC over the month of therapy (UPCday90-UPCday120) and percentage of patients achieving 50% reduction or decrease to <0.5 of UPC with combination therapy.

Conclusions. As shown in FIG. 2, the average change from baseline in UPC was greater from day 30 to day 90 for telmisartan treated dogs compared to enalapril treated dogs with a greater change realized at day 90 for telmisartan treated dogs. The average of UPC change is shown in the following table I

TABLE I
Average of UPC change

| Active | UPC change | UPC change | UPC change |
|---|---|---|---|
| Ingredient | day 30 | day 60 | day 90 |
| Enalapril | −1.075 | −1.260 | −1.194 |
| Telmisartan | −2,516 | −3.769 | −4.341 |

In addition, the average percent change from baseline in UPC is greater in Telmisartan treated dogs compared to Enalapril treated dogs from day 30 to day 90 as shown in FIG. 3. The combination of Telmisartan and Enalapril from day 90 to day 120 achieves a >70% reduction of the average UPC. The average of UPC percent change is shown in the following table II

TABLE II
Average of percent UPC change

| | UPC % | UPC % | UPC % | UPC % |
|---|---|---|---|---|
| Active | change | change | change | change |
| Ingredient | day 30 | day 60 | day 90 | day 120 |
| Enalapril | −27.34 | −37.56 | −30.57 | −75.14¹ |
| Telmisartan | −57.01 | −65.87 | −68.79 | −76.70² |

¹additional telmisartan has been administered between day 90 and day 120
²additional enalapril has been administered between day 90 and day 120

Moreover, the average change in UPC of dogs that received an additional drug at day 90, iwas much greater in dogs initially treated with telmisartan than in the enalapril treatment group as shown in FIG. 4. The average of UPC change is shown in the following table III

TABLE III
Average of UPC change in dogs with added drug at Day 90

| | UPC | UPC | UPC | UPC |
|---|---|---|---|---|
| | change | change | change | change |
| Active Ingredients | day 30 | day 60 | day 90 | day 120 |
| Enalapril¹ | −0.30 | −0.79 | −1.05 | −2.97 |
| Telmisartan² | −4.20 | −4.83 | −4.83 | −5.84 |

¹additional telmisartan has been administered between day 90 and day 120
²additional enalapril has been administered between day 90 and day 120

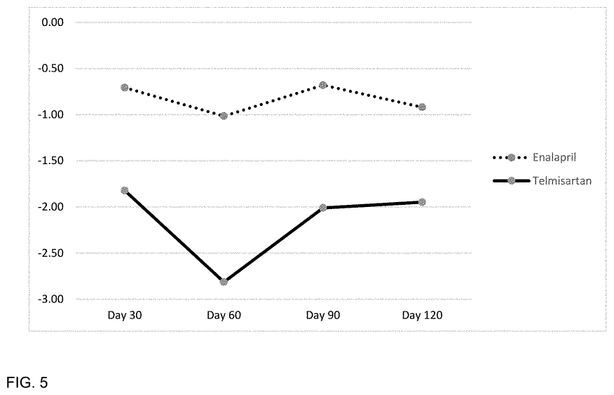

In addition, the average change in UPC that received no additional drug from day 90 to day 120, was also greater in telmisartan treated dogs than in enalapril treated dogs as shown in FIG. 5. The average of UPC change is shown in the following table IV

TABLE IV
Average of UPC change in dogs without added drug at Day 90

| | UPC | UPC | UPC | UPC |
|---|---|---|---|---|
| Active | change | change | change | change |
| Ingredients | day 30 | day 60 | day 90 | day 120 |
| Enalapril | −0.71 | −1.02 | −0.68 | −0.92 |
| Telmisartan | −1.82 | −2.81 | −2.01 | −1.95 |

### REFERENCES

The following publications are hereby incorporated by reference in their entirety as if each individual publication is specifically and individually indicated to be incorporated by reference. In case of conflict, the present application, including any definitions herein, will control.
[1] Brown S. Management of chronic kidney disease In: Elliot J, Grauer G E, eds. BSAVA Manual of Canine and Feline Nephrology and Urology. 2nd ed. Quedgeley, Gloucester: British Small Animal Veterinary Association, 2007; 223-230.
[2] American Veterinary Medical Association. Center for Information Management. U.S. pet ownership & demographics sourcebook. Schaumburg, Ill.: Center for Information Management, 2012.
[3] Polzin D J. Chronic kidney disease in small animals. Vet Clin North Am Small Anim Pract 2011; 41:15-30.
[4] Macdougall D F, Cook T, Steward A P, et al. Canine chronic renal disease: prevalence and types of glomerulonephritis in the dog. Kidney Int 1986; 29: 1144-1151.
[5] Jacob F, Polzin D J, Osborne C A, et al. Evaluation of the association between initial proteinuria and morbidity rate or death in dogs with naturally occurring chronic renal failure. J Am Vet Med Assoc 2005; 226: 393-400.
[6] Walker D S H, Markwell P, Elliott J. Predictors of survival in healthy, non-azotaemic cats (abstract). J Vet Intern Med 2004; 18:417.
[7] Brown S A, Finco D R, Brown C A, et al. Evaluation of the effects of inhibition of angiotensin converting enzyme with enalapril in dogs with induced chronic renal insufficiency. Am J Vet Res 2003; 64: 321327.
[8] Finco D R, Brown S A, Brown C A, et al. Progression of chronic renal disease in the dog. J Vet Intern Med 1999; 13: 516-528.
[9] Lees G E, Brown S A, Elliott J, et al. Assessment and management of proteinuria in dogs and cats: 2004 ACVIM Forum Consensus Statement (small animal). J Vet Intern Med 2005; 19: 377-385.
[10] Valli V E, Baumal R, Thorner P, et al. Dietary modification reduces splitting of glomerular basement membranes and delays death due to renal failure in canine X-linked hereditary nephritis. Lab Invest 1991; 65: 67-73.
[11] Burkholder W J, Lees G E, LeBlanc A K, et al. Diet modulates proteinuria in heterozygous female dogs with X-linked hereditary nephropathy. J Vet Intern Med 2004; 18: 165-175.
[12] Grodecki K M, Gains M J, Baumal R, et al. Treatment of X-linked hereditary nephritis in Samoyed dogs with angiotensin converting enzyme (ACE) inhibitor. J Comp Pathol 1997; 117: 209-225.
[13] Brown S A, Brown C A, Crowell W A, et al. Effects of dietary polyunsaturated fatty acid supplementation in early renal insufficiency in dogs. J Lab Clin Med 2000; 135: 275-286.
[14] Grauer G F, Greco D S, Getzy D M, et al. Effects of enalapril versus placebo as a treatment for canine idiopathic glomerulonephritis. J Vet Intern Med 2000; 14: 526-533.
[15] Brown S A. Renal pathophysiology: lessons learned from the canine remnant kidney model. J Vet Emerg Crit Care (San Antonio) 2013; 23:115-121.
[16] Bugbee A C, Coleman A E, Wang A, Woolcock A D, Brown S A, J Vet Intern Med 2014; 28: 1871-1874.
[17] Kwon Y-J, Suh G-H, Kang S-S, Kim H-J, Can Vet J 2018; 59: 759-762.

1. Telmisartan or a pharmaceutically acceptable salt thereof for use in a method for the treatment of elevated urinary protein-to-creatinine ratio (UPC) levels in a dog in need of such treatment, wherein the method comprises administration of a therapeutically effective amount of telmisartan to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

2. Telmisartan according to claim 1, wherein the elevated UPC levels are associated with chronic kidney disease (CKD).

3. Telmisartan according to claim 1 or 2, wherein the elevated UPC levels are associated with protein losing nephropathy (PLN).

4. Telmisartan according to any one of claims 1 to 3, wherein CKD and the elevated UPC levels are associated with systemic hypertension.

5. Telmisartan according to any one of claims 1 to 4, which is the sodium or potassium salt thereof.

6. Telmisartan according to any one of claims 1 to 5, wherein the daily therapeutically effective amount thereof ranges from 1.0 to 4.0 mg/kg of body weight.

7. Telmisartan according to any one of claims 1 to 6, wherein the daily dosage amount of telmisartan is increased for the second period of time by an incremental amount ranging from 0.25 to 2.50 mg/kg of body weight.

8. Telmisartan according to any one of claims 1 to 7, wherein the daily dosage amount of telmisartan for a first period of time during the treatment period is 1.0 to 1.5 mg/kg of body weight, and the daily dosage amount of telmisartan for the second period of time is 1.75 to 3.50 mg/kg of body weight.

9. Telmisartan according to any one of claims 1 to 8, wherein the daily dosage amount of telmisartan is decreased after the second period of time by an incremental amount ranging from 0.25 to 2.50 mg/kg of body weight.

10. Telmisartan according to any one of claims 1 to 9, wherein the daily dosage amount of telmisartan is decreased after the second period of time when the urinary protein-to-creatinine ratio (UPC) level measured for the dog decreases by at least 70% in relation to a baseline UPC value measured for the dog prior to the first period of time.

11. Telmisartan according to any one of claims 1 to 8, which is administered together with at least one other drug to a dog in need of such a treatment.

12. Telmisartan according to claim 9, wherein the other drug is selected from the group consisting of calcium channel blockers, preferably amlodipine, cardiotonic-calcium sensitizing agents, preferably pimobendan or levosimendan, ACE inhibitors, preferably ramipril, benazepril or enalapril.

13. Telmisartan according to any one of claims 1 to 9, wherein the UPC level is decreased by at least 50% compared to the baseline within the first period of treatment.

14. Telmisartan or a pharmaceutically acceptable salt thereof as a medicament for the treatment of diseases or disorders associated with elevated urinary protein-to-creatinine ratio (UPC) levels, which are non-refractory to the treatment with ACE inhibitors in dogs.

15. A pharmaceutical composition comprising telmisartan or a pharmaceutically acceptable salt thereof and a pharmaceutically acceptable carrier for use in a method for the treatment of d elevated urinary protein-to-creatinine ratio (UPC) levels, in a dog in need of such treatment, wherein the method comprises administration of a therapeutically effective amount of telmisartan to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

16. A method for the treatment of elevated urinary protein-to-creatinine ratio (UPC) levels, in a dog in need of such treatment, wherein the method comprises administration of a therapeutically effective amount of telmisartan or a pharmaceutically acceptable salt thereof to the dog, wherein the therapeutically effective amount of telmisartan is administered in a daily dosage amount that is varied over a treatment period, the daily dosage amount of telmisartan for a first period of time during the treatment period is at least 1.0 mg/kg of body weight, and the daily dosage amount of telmisartan is increased for a second period of time subsequent the first period of time during the treatment period.

17. The method according to claim 16, wherein the elevated UPC levels are associated with chronic kidney disease (CKD).

18. The method according to claim 16, wherein the elevated UPC levels are associated with of protein losing nephropathy (PLN).

19. The method according to claim 16, wherein the elevated UPC levels are associated with CKD and PLN.

20. The method according to claim 16, wherein the elevated UPC levels are associated with systemic hypertension.

21. The method according to claim 16, which comprises administration of an effective amount of the sodium or potassium salt of telmisartan.

22. The method according to claim 16, wherein the daily therapeutically effective amount of telmisartan ranges from 1.0 to 4.0 mg/kg of body weight.

23. The method according to claim 16, wherein the daily dosage amount of telmisartan is increased for the second period of time by an incremental amount ranging from 0.25 to 2.50 mg/kg of body weight.

24. The method according to claim 16, wherein the daily dosage amount of telmisartan is decreased after the second period of time by an incremental amount ranging from 0.25 to 2.50 mg/kg of body weight.

25. The method according to claim 24, wherein the daily dosage amount of telmisartan is decreased after the second period of time when the urinary protein-to-creatinine ratio (UPC) level measured for the dog decreases by at least 70% in relation to a baseline UPC value measured for the dog prior to the first period of time.

26. The method according to claim 16, wherein the method further comprises administration of at least one other drug to such dog in need of such a treatment.

27. The method according to claim 26, wherein the other drug is selected from the group consisting of calcium channel blockers, cardiotonic-calcium sensitizing agents and ACE inhibitors.

28. The method according to claim 27, wherein the other drug is selected from the group consisting of amlodipine, pimobendan, levosimendan, ramipril, benazepril and enalapril.

29. The method according to claim 16, wherein the UPC level is decreased in by at least 50 compared to the baseline within the first period of treatment.

30. A method for the treatment of diseases or disorders, which are associated with elevated urinary protein-to-creatinine ratio (UPC) levels, which are non-refractory to the treatment with ACE inhibitors in dogs, which method comprises administration of a therapeutically effective amount of telmisartan or a pharmaceutically acceptable salt thereof to a dog in need of such a treatment.
